(19) United States
(12) Patent Application Publication
(10) Pub. No.: US 2023/0405006 A1
(43) Pub. Date: Dec. 21, 2023

(54) COMBINATION PHARMACEUTICAL COMPOSITION AND TREATMENT METHOD

(71) Applicant: CARNA BIOSCIENCES, INC., Hyogo (JP)

(72) Inventors: Shigeki KASHIMOTO, Kobe-shi, Hyogo (JP); Masaaki SAWA, Kobe-shi, Hyogo (JP)

(73) Assignee: CARNA BIOSCIENCES, INC., Hyogo (JP)

(21) Appl. No.: 18/035,592

(22) PCT Filed: Nov. 11, 2021
(86) PCT No.: PCT/JP2021/041571

§ 371 Date: May 5, 2023

(30) Foreign Application Priority Data
Nov. 13, 2020 (JP) 2020-189532

(51) Int. Cl. A61K 31/519; A61K 39/395; A61K 31/495; A61P 35/00

(52) U.S. Cl. CPC A61K 31/519; A61K 39/3955; A61K 31/495; A61P 35/00; A61K 2039/505

(57) ABSTRACT

Provided is a novel cancer treatment means in which a reversible BTK inhibitor and immunity checkpoint inhibitor are combined. For example, provided is a pharmaceutical composition for treating cancer wherein BTK inhibitor (I-A) and anti PD-1 antibody are combined.

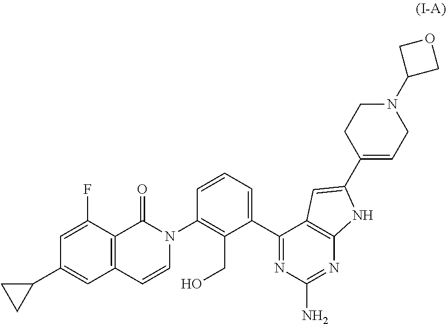 [SMILES: Nc1nc(-c2cccc(-n3ccc4cc(C5CC5)cc(F)c4c3=O)c2CO)c2cc(C3=CCN(C4COC4)CC3)[nH]c2n1]

### TECHNICAL FIELD

The present invention relates to a pharmaceutical composition for treating a cancer containing a reversible BTK inhibitor and an immune checkpoint inhibitor in combination, and a method for treating a cancer with use of the composition.

### BACKGROUND ART

Bruton's tyrosine kinase (BTK) is a member of the Tec family of non-receptor tyrosine kinases and is an important signaling enzyme expressed in all hematopoietic cell types except T lymphocytes and natural killer cells. BTK is an important regulator of survival, differentiation, proliferation, activation, and the like of B cells, and plays an important role in B cell signaling (Non Patent Documents 1 and 2). B-cell receptors (BCRs) on the cell surface transmit signals into the cell via BTK present downstream thereof. Thus, abnormal activation of the B cell signaling pathway is considered to promote proliferation and survival of cancer cells of B-cell lymphoma, chronic lymphocytic leukemia and the like (Non Patent Document 3). BTK is also known to play an important role in signaling pathways of many other cells and is deemed to be involved in allergic diseases, autoimmune diseases and inflammatory diseases (Non Patent Document 1). For example, BTK plays an important role in signaling of high affinity IgE receptors (FcεRI) in mast cells, and BTK-deficient mast cells are known to have reduced degranulation and decreased production of proinflammatory cytokines (Non Patent Document 4). The irreversible BTK inhibitor drug ibrutinib is an anticancer agent used in the treatment of B-cell tumors. In recent years, it has been revealed that C481S mutation in BTK results in ibrutinib resistance during ibrutinib treatment (Non Patent Document 5). More recently, an isoform p65BTK has been reported to be expressed downstream of RAS signals in solid cancers other than hematological cancers and to have a profound effect on proliferation in solid cancers such as colon cancer cells (Non Patent Document 6). Thus, compounds having BTK inhibitory activity are considered to be useful for the treatment of diseases in which BTK signals are involved, such as cancer, B-cell lymphoma and chronic lymphocytic leukemia, and also useful for the treatment of solid cancers in which p65BTK is expressed. Meanwhile, oxoisoquinoline and triazine derivatives having reversible BTK inhibitory actions have been reported as reversible BTK inhibitor drugs that are effective for cancers that have mutations in BTK and are resistant to irreversible BTK inhibitors such as ibrutinib (Patent Documents 1 and 2).

On the other hand, cancer immunotherapies that achieve antitumor effect by enhancing the antitumor immune response of a host with an immune checkpoint inhibitor such as an anti-CTLA-4 (Cyototoxic T lymphocyte antigen 4) antibody, an anti-PD-1 (Programmed death receptor 1) antibody, and an anti-PD-L1 (Programmed death ligand 1) antibody have been recently gathering attention for treating cancers. Several immune checkpoint inhibitors have been approved as pharmaceuticals so far and their antitumor effects have been confirmed, whereas the immune checkpoint inhibitors have been reported to be effective for a limited number of patients, and even cause some patients to have resistance thereto (Non Patent Document 7).

BTK inhibitors have been recently reported to improve tumor immunity and synergistically exhibit antitumor effect when being used in combination with an immune checkpoint inhibitor (Non Patent Documents 8, 9, and 10). Accordingly, BTK inhibitors are not only useful as cancer immunotherapy for treating cancers through immunostimulation by activating immunocytes, but also useful for extension and enhancement of therapeutic effect in conventional cancer immunotherapies through the use of an immune checkpoint inhibitor or the like in combination.

In addition, chimeric antigen receptor-expressing T cell (CAR-T cell) therapy has been gathering attention as one of cancer immunotherapies without use of an antibody. However, the CAR-T cell therapy exerts high therapeutic effect to blood cancers, but has not achieved satisfactory effect to solid cancers. BTK inhibitors have been reported to enhance the functions of CAR-T cells to give increased antitumor effect (Non Patent Document 11). Accordingly, when being used in combination with CAR-T cell therapy, BTK inhibitors are useful for extension and enhancement of the therapeutic effect.

While activation of tumor immunity with the irreversible BTK inhibitor ibrutinib and the combination of ibrutinib and cancer immunotherapy have been reported, the combination of a reversible BTK inhibitor and cancer immunotherapy according to the present invention has not been disclosed at all, and enhancement of antitumor effect by the combination of a reversible BTK inhibitor and an immune checkpoint inhibitor according to the present invention has never been reported.

### PRIOR ART DOCUMENTS

| | |
|---|---|
| PATENT | |
| DOCUMENTS | |
| [Patent | WO 2018/097234 |
| Document 1] | |
| [Patent | WO 2015/012149 |
| Document 2] | |
| NON PATENT | |
| DOCUMENTS | |
| [Non Patent | Satterthwaite, A. B. and |
| Document 1] | Witte, O. N., Immunol. Rev., 2000, |
| | 175, 120-127. |
| [Non Patent | Kurosaki, T., |
| Document 2] | Curr. Opin. Immunol., 2000, 12, 276-281. |
| [Non Patent | Davis, R. E., et al., Nature, |
| Document 3] | 2010, 463, 88-92. |
| [Non Patent | Ellmeier, W., et al., FEBS J., |
| Document 4] | 2011, 278, 1990-2000. |
| [Non Patent | Cheng, S., et al., Leukemia, |
| Document 5] | 2014, 1-6. |
| [Non Patent | Grassili. E., et al., Oncogene, |
| Document 6] | 2016, 35, 4368-4378. |
| [Non Patent | Spranger, S. and Gajewski, T. F., |
| Document 7] | Nat. Rev. Cancer., 2018, 18, 139-147. |
| [Non Patent | Sagiv-Barfi, I., et al., Proc. |
| Document 8] | Natl. Acad. Sci. U.S.A,. 2015, 112, |
| | E966-E972. |
| [Non Patent | Gunderson, A. J., et al., Cancer |
| Document 9] | Discov., 2016, 6, 270-285. |
| [Non Patent | Stiff, A., et al., Cancer Res., |
| Document 10] | 2016, 76, 2125-2136. |
| [Non Patent | Wang, L., et al., Int. |
| Document 11] | Immunopharmacol., 2019, 70, 498-503. |

### SUMMARY OF INVENTION

### Problem to be Solved by the Inventions

An objective of the present invention, which is based on the finding of a more effective cancer treatment method by combining an immunological treatment method for a cancer and a reversible BTK inhibitor, is to provide with a novel pharmaceutical composition for treating a cancer.

### Means for Solving the Problems

The present inventors diligently studied to solve the above problems, and have found that a combination of a compound represented by a general formula (I) or (II) described later or a pharmaceutically acceptable salt thereof and an immune checkpoint inhibitor can solve the problems to be solved by the present invention, and eventually completed the present invention.

Specifically, the present invention relates to follows;
(1) A pharmaceutical composition for treating a cancer comprising a reversible BTK inhibitor and an immune checkpoint inhibitor in combination;
(2) A pharmaceutical composition described in (1), wherein the reversible BTK inhibitor is an oxoisoquinoline derivative of the following formula (I)

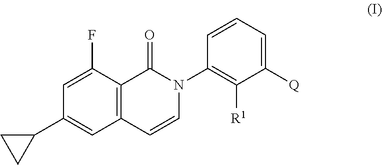

wherein R¹ is an optionally substituted lower alkyl group, Q is a structure selected from the following structures (a), (b) and (c);

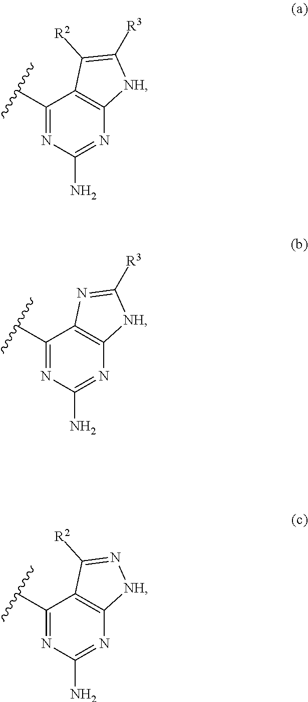

R² and R³ are independently a hydrogen atom, an optionally substituted lower alkyl group, an optionally substituted cycloalkyl group, an optionally substituted aryl group, an optionally substituted heteroaryl group or an optionally substituted heterocyclic group,
   or a pharmaceutically acceptable salt thereof;
(3) the pharmaceutical composition described in (2), wherein Q is a structure (a), and R¹ is a hydroxymethyl group;
(4) the pharmaceutical composition described in (1), wherein the reversible BTK inhibitor is an oxoisoquinoline derivative of the following formula (Ia)

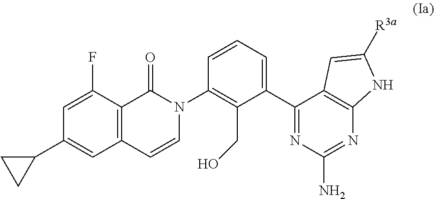

wherein R^{3a} is an optionally substituted tetrahydropyridyl group,
   or a pharmaceutically acceptable salt thereof;
(5) the pharmaceutical composition described in (4), wherein the oxoisoquinoline derivative has a structure of Compound (I-A);

### Compound (I-A): 2-(3-{2-amino-6-[1-(oxetan-3-yl)-1,2,3,6-tetrahydropyridin-4-yl]-7H-pyrrolo[2,3-d]pyrimidin-4-yl}-2-(hydroxymethyl)phenyl)-6-cyclopropyl-8-fluoroisoquinolin-1(2H)-one

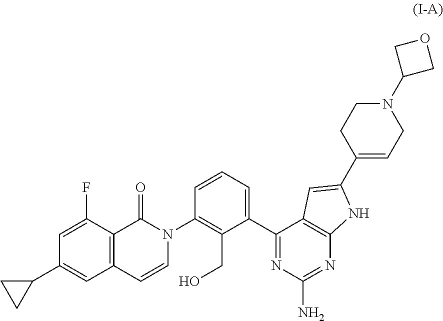 [SMILES: Nc1nc(-c2cccc(-n3ccc4cc(C5CC5)cc(F)c4c3=O)c2CO)c2cc(C3=CCN(C4COC4)CC3)[nH]c2n1] [0020] (6) the pharmaceutical composition described in (1), wherein the reversible BTK inhibitor is a triazine derivative of the following formula (II)

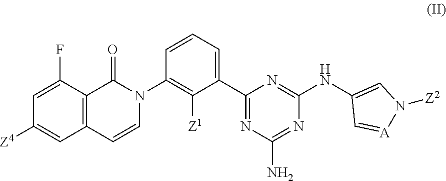

wherein Z¹ represents an optionally substituted lower alkyl group,
   Z² represents a hydrogen atom or an optionally substituted lower alkyl group,
   A represents a nitrogen atom or C-Z³,
   Z³ represents a hydrogen atom, a cyano group, an optionally substituted acyl group, an optionally substituted sulfonyl group, or an optionally substituted carbamoyl group, Z⁴ represents an optionally substituted lower alkyl group, an optionally substituted cycloalkyl group,
   or a pharmaceutically acceptable salt thereof;
(7) the pharmaceutical composition described in (6), wherein Z¹ is a hydroxymethyl group;
(8) the pharmaceutical composition described in (6), wherein the triazine derivative has a structure of Compound (II-A);

### Compound (II-A): 2-(3-{4-Amino-6-[(1-methyl-1H-pyrazol-4-yl)amino]-1,3,5-triazin-2-yl}-2-(hydroxymethyl)phenyl)-6-cyclopropyl-8-fluoroisoquinolin-1(2H)-one

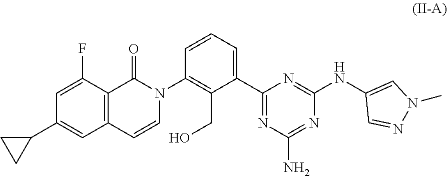 [SMILES: Cn1cc(Nc2nc(N)nc(-c3cccc(-n4ccc5cc(C6CC6)cc(F)c5c4=O)c3CO)n2)cn1] [0025] (9) the pharmaceutical composition described in any of (1) to (8), wherein the immune checkpoint inhibitor is an inhibitor to immune checkpoint molecules selected from the group consisting of PD-1, PD-L1, PD-L2, CTLA-4, LAG-3, TIM3, BTLA, B7H3, B7H4, CD160, CD39, CD73, A2aR, KIR, VISTA, IDO1, ArginaseI, TIGIT and CD115;
(10) the pharmaceutical composition described in (9), wherein the immune checkpoint inhibitor is an anti-PD-1 antibody;
(11) the pharmaceutical composition described in (9), wherein the immune checkpoint inhibitor is an anti-PD-L1 antibody;
(12) the pharmaceutical composition described in (9), wherein the immune checkpoint inhibitor is an anti-PD-L2 antibody;
(13) the pharmaceutical composition described in (9), wherein the immune checkpoint inhibitor is an anti-CTLA-4 antibody;
(14) Use of a compound (I) and an immune checkpoint inhibitor in manufacturing the pharmaceutical composition described in (1); and
(15) Method for treating a cancer characterized in using the pharmaceutical composition described in any one of (1) to (13).

### Effect of the Invention

The combination of a reversible BTK inhibitor and an immune checkpoint inhibitor can result in anticancer action with higher efficiency than when the BTK inhibitor or the immune checkpoint inhibitor each is used alone. Thus, the combination of the BTK inhibitor and the immune checkpoint inhibitor can be expected to exhibit synergistic effects as anticancer action and is useful as a prophylaxis or treatment of cancer and the like.

### BRIEF DESCRIPTION OF DRAWINGS

FIG. 1 shows that a reversible BTK inhibitor (I-A), alone or in combination with an anti-PD-1 antibody, suppresses tumor growth in an allograft mouse model with the mouse colorectal cancer cell line CT26.WT (Example 1).

FIG. 2 shows that a reversible BTK inhibitor (I-A) in combination with an anti-PD-1 antibody gives high antitumor effect in an allograft mouse model with the mouse B-cell lymphoma cell line A20 (Example 2).

### BEST MODE FOR CARRYING OUT THE INVENTION

### (1) Reversible BTK Inhibitors

One of embodiments in the reversible BTK inhibitors is an oxoisoquinoline derivative represented by the following formula (I) described in WO2018/097234 (Patent Document 1);

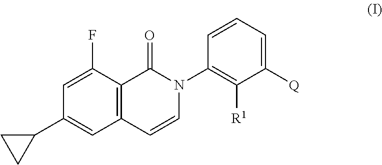

wherein R¹ is an optionally substituted lower alkyl group, Q is a structure selected from the following structures (a), (b) and (c);

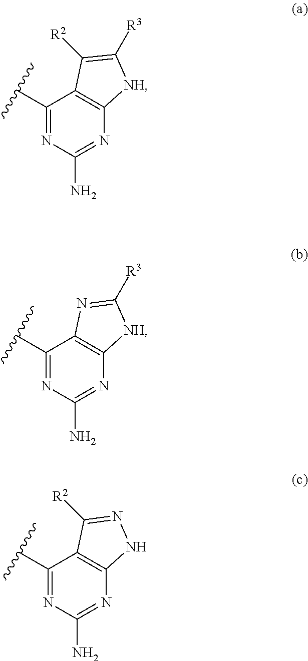

R² and R³ are independently a hydrogen atom, an optionally substituted lower alkyl group, an optionally substituted cycloalkyl group, an optionally substituted aryl group, an optionally substituted heteroaryl group or an optionally substituted heterocyclic group, or a pharmaceutically acceptable salt thereof.

In the specification formula (I) of the present application, a moiety of the lower alkyl group in the “optionally substituted lower alkyl group” may be any of a linear, or branched alkyl group having one to three carbon atoms, and specifically a methyl group, an ethyl group, and an isopropyl group etc. may be exemplified.

A moiety of the cycloalkyl group in the “optionally substituted cycloalkyl group” may be any of cyclic alkyl group having three to six carbon atoms, and specifically a cyclopropyl group, a cyclobutyl group, cyclohexyl group etc. may be exemplified.

A moiety of the aryl group in the “optionally substituted aryl group” may be any of monocyclic or bicyclic aryl group having 6 to 14 carbon atoms, and the bicyclic aryl group may be partially hydrogenated. Specifically, a phenyl group, a naphthyl group, a tetrahydronaphthyl group, an indenyl group etc. may be exemplified.

A moiety of the heteroaryl group in the “optionally substituted heteroaryl group” includes a monocyclic aromatic heterocyclic group and a fused aromatic heterocyclic group, and 5- or 6-membered monocyclic aromatic heterocyclic group containing one heteroatom at least selected from a nitrogen atom, a sulfer atom and an oxygen atom as the monocyclic aromatic heterocyclic group. Specifically, pyrrolyl, imidazolyl, pyrazolyl, thienyl, thiazolyl, furanyl, pyridyl, pyrimidyl, pyridazyl etc. may be exemplified, and examples of the fused aromatic heterocyclic group include a fused bicyclic heterocyclic group in which 3- to 8-membered ring is fused containing one heteroatom at least selected from a nitrogen atom, a sulfer atom and an oxygen atom. Specifically tetrahydroisoquinolyl, benzothiophenyl, benzimidazolyl, benzooxazolyl, benzothiazolyl, indolyl, and isoquinolyl may be exemplified.

A moiety of the heterocyclic group in the “optionally substituted hetercyclic group” is a 4- to 6-membered monocyclic saturated heterocyclic group containing one heteroatom at least selected from a nitrogen atom, a sulfer atom and an oxygen atom and may include an unsaturated bond partially in the ring. Specifically, a dihydrothiopyranyl group, 1,1-dioxo-dihydrothiopyranyl group, and tetrahydropyridyl group may be exemplified, and the tetrahydropyridyl group is especially preferably exemplified.

A substituent of the term of “optionally substituted” in the optionally substituted lower alkyl group, the optionally substituted cycloalkyl group, the optionally substituted aryl group, the optionally substituted heteroaryl group, and the optionally substituted heterocyclic group may be the same or different when the above group have two or more substituents, and the group may be substituted with one, or two or more of any kind of substituent(s) at any position which is chemically allowable.

Examples of the substituent in the optionally substituted lower alkyl group include for example, a halogen atom, a C1-C4 alkoxy group, an amino group optionally substituted with one or two C1-C4 alkyl group, a nitro group, a cyano group, a hydroxy group, a carbamoyl group optionally substituted with one or two C1-C4 alkyl group, a carboxyl group, a formyl group, an acetyl group, a mesyl group, a benzoyl group, a C1-C6 acylamino group, a C1-C6 acyloxy group etc.

As the optionally substituted lower alkyl group, a hydroxymethyl group may be exemplified.

Examples of a substituent related to the term of “optionally substituted” in the optionally substituted cycloalkyl group, the optionally substituted aryl group, the optionally substituted heteroaryl group and the optionally substituted heterocyclic group include a halogen atom, an oxygen atom, a C1-C4 alkyl group, a C1-C4 alkoxy group, an amino group optionally substituted with one or two C1-C4 alkyl group, a nitro group, a cyano group, a hydroxy group, a carbamoyl group optionally substituted with one or two C1-C4 alkyl group, a sulfonyl group optionally substituted with a C1-C4 alkyl group, a carboxy group, a formyl group, an acetyl group, a mesyl group, a benzoyl group, an oxetanyl group, a C1-C6 acylamino group, and a C1-C6 acyloxy group etc.

Isomers may exist in the compound (I) of the present invention, depending on the kind of the substituent. In the present specification, the isomers may be described by a chemical structure of only one form thereof, but the present invention includes all isomers (geometrical isomer, optical isomer, tautomer, etc.) which can be structurally formed, and also includes isomers alone, or a mixture thereof.

Examples of a pharmaceutically acceptable salt of the compound (I) of the present invention include inorganic acid salts with hydrochloric acid, sulfuric acid, carbonic acid, and phosphoric acid etc.; and organic acid salts with fumaric acid, maleic acid, methanesulfonic acid, and p-toluenesulfonic acid etc. The present invention also includes ammonium salts, in addition to alkali metal salts with sodium and potassium; alkaline earth metal salts with magnesium and calcium; organic amine salts with triethylamine and ethanolamine; and basic amino acid salts with lysine, arginine, and ornithine etc.

The compound (I) and a pharmaceutically acceptable salt thereof in the present invention can be produced, for example, by methods described in Patent Document 1. When a defined group may be chemically affected under the conditions of an exemplified method in the production method shown below, or is unsuited for use to carry out the method, it is possible to easily produce them by a method which is usually used in organic synthetic chemistry, for example, a method of applying means such as protection or deprotection of a functional group [T. W. Greene, Protective Groups in Organic Synthesis 3rd Edition, John Wiley&Sons, Inc., 1999]. If necessary, the order of a reaction step such as introduction of substituents can also be changed.

Preferably Q is a structure (a) and R¹ is a hydroxymethyl group in the compound (I) above, and more preferably, the compound (I) is Compound (I-A): 2-(3-{2-amino-6-[1-(oxetan-3-yl)-1,2,3,6-tetrahydropyridin-4-yl]-7H-pyrrolo[2,3-d]pyrimidin-4-yl}-2-(hydroxymethyl)phenyl)-6-cyclopropyl-8-fluoroisoquinolin -1(2H)-one.

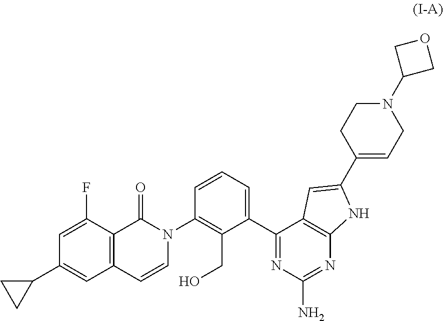 [SMILES: Nc1nc(-c2cccc(-n3ccc4cc(C5CC5)cc(F)c4c3=O)c2CO)c2cc(C3=CCN(C4COC4)CC3)[nH]c2n1]

Additionally, Compound (I-A) is a compound of example 23 in the Patent Document 1.

Another embodiment of the reversible BTK inhibitor is a triazine derivative represented by the following formula (II) described in WO2015/012149 (Patent Document 2);

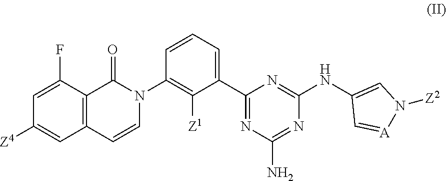

wherein Z¹ represents an optionally substituted lower alkyl group,
   Z² represents a hydrogen atom or an optionally substituted lower alkyl group,
   A represents a nitrogen atom or C-Z³ ,
   Z³ represents a hydrogen atom, a cyano group, an optionally substituted acyl group, an optionally substituted sulfonyl group, or an optionally substituted carbamoyl group, Z⁴ represents an optionally substituted lower alkyl group, an optionally substituted cycloalkyl group,
   or a pharmaceutically acceptable salt thereof.

In the compound (II), a lower alkyl group moiety of the optionally substituted lower alkyl group may be any of linear, branched or cyclic alkyl groups having 1 to 3 carbon atoms, and specific examples thereof include a methyl group and an isopropyl group, etc.

A cycloalkyl group moiety of the optionally substituted cycloalkyl group may be cyclic alkyl groups having 3 to 6 carbon atoms, and specific examples thereof include a cyclopropyl group and a cyclobutyl group, etc.

An acyl group moiety of the optionally substituted acyl group may be any of linear, branched or cyclic alkyl groups connected to a carbonyl group, and specific examples of the acyl group moiety of the optionally substituted acyl group include a formyl group, an acetyl group and a propionyl group, an octanoyl group, a dodecanoyl group, a pivaloyl group, a cyclopropylcarbonyl group and a benzoyl group etc.

Examples of the sulfonyl group moiety of the optionally substituted sulfonyl group include a methylsulfonyl group, an ethylsulfonyl group, etc.

Examples of the carbamoyl group moiety of the optionally substituted carbamoyl group include a methylcarbamoyl group, an ethylcarbamoyl group and a dimethylcarbamoyl group, etc.

A substituent of the optionally substituted lower alkyl group, the optionally substituted cycloalkyl group, the optionally substituted acyl group, the optionally substituted sulfonyl group, or the optionally substituted carbamoyl group may be the same or different when the above group have two or more substituents, and the group may be substituted with one, or two or more of any kind of substituent(s) at any position which is chemically allowable. Examples of the substituent include a halogen atom, a substituted or unsubstituted alkyl group, a substituted or unsubstituted alkoxy group, a substituted or unsubstituted amino group, a nitro group, a cyano group, a hydroxy group, a substituted or unsubstituted alkylamino group, a substituted or unsubstituted carbamoyl group, a carboxyl group, a formyl group, an acetyl group, a mesyl group, a benzoyl group, a substituted or unsubstituted acylamino group, and a substituted or unsubstituted acyloxy group, etc.

Isomers may exist in the compound (II) of the present invention, depending on the kind of the substituent. In the present description, the isomers may be described by a chemical structure of only one form thereof, but the present invention includes all isomers (geometrical isomer, optical isomer, tautomer, etc.) which can be structurally formed, and also includes isomers alone, or a mixture thereof.

Examples of the pharmaceutically acceptable salt of the compound (II) of the present invention include inorganic acid salts with hydrochloric acid, sulfuric acid, carbonic acid, and phosphoric acid; and organic acid salts with fumaric acid, maleic acid, methanesulfonic acid, and p-toluenesulfonic acid. The present invention also includes ammonium salts, in addition to alkali metal salts with sodium and potassium; alkaline earth metal salts with magnesium and calcium; organic amine salts with lower alkylamine and lower alcoholamine; and basic amino acid salts with lysine, arginine, and ornithine.

The compound (II) and a pharmaceutically acceptable salt thereof in the present invention can be produced, for example, by methods described in Patent Document 2. When a defined group may be chemically affected under the conditions of an exemplified method in the production method shown below, or is unsuited for use to carry out the method, it is possible to easily produce them by a method which is usually used in organic synthetic chemistry, for example, a method of applying means such as protection or deprotection of a functional group [T. W. Greene, Protective Groups in Organic Synthesis 3rd Edition, John Wiley&Sons, Inc., 1999]. If necessary, the order of a reaction step such as introduction of substituents can also be changed.

Preferably, A is a nitrogen atom and Z¹ is a hydroxymethyl group in the compound (II), and more preferably the compound (II) is Compound (II-A): 2-(3-{4-amino-6-[(1-methyl-1H-pyrazol-4-yl)amino]-1,3,5-triazin -2-yl}-2-(hydroxymethyl)phenyl)-6-cyclopropyl-8-fluoroisoquinolin-1(2H)-one

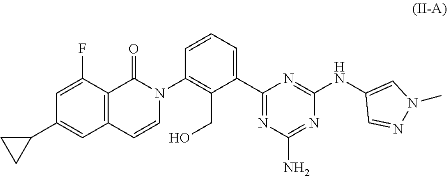 [SMILES: Cn1cc(Nc2nc(N)nc(-c3cccc(-n4ccc5cc(C6CC6)cc(F)c5c4=O)c3CO)n2)cn1]

Additionally, Compound (II-A) is a compound of example 1 in the Patent Document 2.

### (2) Immune Checkpoint Inhibitors

In the present invention, the immune checkpoint inhibitor refers to a substance that inhibits the functions of immune checkpoint molecules acting on the immune checkpoint system, and may be any substance having immune checkpoint inhibitory action, without limitation.

The immune checkpoint molecules may be any molecules controlling the immune checkpoint system, without limitation, and examples thereof include immune checkpoint molecules known from documents and the like (e.g., see Qin et al., Molecular Cancer, 2019, 18, 155), specifically, PD-1, PD-L1, PD-L2, CTLA-4, LAG-3, TIM3, BTLA, B7H3, B7H4, CD160, CD39, CD73, A2aR, KIR, VISTA, IDO1, ArginaseI, TIGIT and CD115.

Examples of the immune checkpoint inhibitor include, but are not limited to, substances that inhibit the functions of human immune checkpoint molecules such as neutralizing antibodies.

Specific examples are anti-PD-1 antibodies (e.g., nivolumab, pembrolizumab), anti-PD-L1 antibodies (e.g., avelumab, atezolizumab, durvalumab), anti-PD-L2 antibodies, and anti-CTLA-4 antibodies (e.g., ipilimumab, tremelimumab).

In the present invention, any one or more of those immune checkpoint inhibitors can be used in combination with the reversible BTK inhibitor to be used for the present invention.

### (3) Combination Pharmaceutical Composition

The pharmaceutical composition according to the present invention is a composition that appropriately combines a reversible BTK inhibitor and an immune checkpoint inhibitor, and encompasses a kit including the BTK inhibitor and the immune checkpoint inhibitor. Both inhibitors may be administered concurrently together as a formulation of a mixture or as separate formulations. Alternatively, they may be administered in any order sequentially or with appropriate intervals as separate formulations.

The doses of the reversible BTK inhibitor and the immune checkpoint inhibitor to be used in the present invention can vary according to the method of administration (oral, parenteral, or topical administration), the type of disease to which they are applied, the severity of the disease, the age and weight of the patient, or the like.

The dose of the reversible BTK inhibitor to be used in the present invention is typically in the range of 0.01 mg to 1,000 mg per day in adults, and it can be administered by oral or parenteral route once or two or three times by divided dosage.

The dose of the immune checkpoint inhibitor to be used for the combination of the present invention, which varies depending on the dose of the reversible BTK inhibitor to be used in the present invention, the method of administration (oral, parenteral, or topical administration), the type of disease to which they are applied, the severity of the disease, the age and weight of the patient, or the like, can be adjusted so as to achieve the desired optimum effect, typically being in the range of 0.1 mg to 20 mg/kg per day in adults, and the immune checkpoint inhibitor is administered by parenteral route (e.g., intravenous continuous administration).

Concomitant administration of the same dosage form or different dosage forms, or separate administration is applicable as a method of combinatorial administration for the combination of the present invention.

The pharmaceutical composition containing the combination of the present invention is useful for treating a cancer, and examples of the cancer include blood cancers (e.g., leukemia, malignant lymphoma, multiple myeloma, myelodysplastic syndrome), solid cancers (e.g., gastric cancer, colorectal cancer, lung cancer, esophageal cancer, liver cancer, breast cancer, ovarian cancer, uterine cancer, renal cancer, prostate cancer, skin cancer), osteosarcoma, mesothelioma, and cancer of unknown primary.

The pharmaceutical composition containing the combination of the present invention is expected to exert antitumor effect to cancer patients for whom the therapeutic effect of a reversible BTK inhibitor or an immune checkpoint inhibitor alone is unsatisfactory, and also expected to mitigate side effects and the like because the enhanced antitumor effect provided by the combination of the present invention allows the doses of the agents to be reduced.

In addition, the combination of the present invention is expected to be effective for prevention of the metastasis and recurrence of cancers, and even for treatment of metastatic cancers.

### EXAMPLES

### Example 1 Combined Effect of Reversible BTK Inhibitor and Anti-PD-1 Antibody in Allograft Mouse Model with Mouse Colorectal Cancer Cell Line CT26.WT

The antitumor effect of the compound according to the present invention was examined by using a syngeneic mouse tumor model with the mouse colorectal cancer cell line CT26.WT (subcutaneous transplantation).

### (Culture of Cells for Use)

To RPMI-1640 medium (Life Technologies, No. A1049101), 10% FBS (Biowest) and 1% penicillin streptomycin (NACALAI TESQUE, INC.) were added to prepare a cell culture medium (hereinafter, referred to as the medium 1). CT26.WT cells (ATCC) were cultured with the medium 1 in a flask in a 5% CO₂ incubator.

### (Production of Cancer-Bearing Model)

The cell density of CT26.WT cells was adjusted with RPMI-1640 medium (containing 1% penicillin streptomycin, hereinafter, referred to as the medium 2) to 5×10⁶ cells/mL; thus a cell preparation solution for transplantation was produced. Into the back of each BALB/cCrslc mouse (female, 8-week-old, Japan SLC, Inc.), 0.1 mL of the cell preparation solution for transplantation was subcutaneously transplanted. On day 3 after the transplantation of cancer cells, the cancer-bearing mice were grouped in such a manner that the means of tumor volume (see a calculation formula below) approximated to each other.

### (Preparation of Sample Solution for Administration of Test Substance)

As a sample solution for administration of the test substance, a 1.5 mg/mL solution was prepared. The test substance (71.9 mg) was dissolved in DMSO (2.4 mL, NACALAI TESQUE, INC.), to which polyethylene glycol #400 (24.0 mL, NACALAI TESQUE, INC.) was added and mixed well, and 21.5 mL of 30% (w/v) aqueous solution of 2-hydroxypropyl-β-cyclodextrin (HP-β-CD, Wacker Chemical AG) was then added thereto to produce a 1.5 mg/mL sample solution for administration.

### (Preparation of Antibody Solution)

An anti-PD-1 antibody (manufactured by Bio X cell, clone RMP1-14; catalog No. BE0146) was prepared as a 1 mg/mL solution with physiological saline immediately before administration.

### (Antitumor Effect Test for Test Substance)

Forced oral administration of 0.1 mL of the test substance or a solvent per 10 g body weight on that day (15 mg/kg) was performed to each mouse with transplanted cancer cells (six mice per group) twice per day (interval: 6 hours or longer) from day 3 to day 21 after the transplantation. No administration was performed on day 8, 9, 15, and 16 after the transplantation. To each mouse, 0.1 mL of the antibody solution per 10 g body weight on that day (10 mg/kg) was intraperitoneally administered for an antibody administration group and a combined agents group, and the same amount of physiological saline was intraperitoneally administered for the other groups twice per week (six times in total). The mice were followed up until day 48 after the transplantation, and the tumor diameter of each mouse was measured to calculate the tumor volume by using the following formula several times per week for evaluation of the antitumor effect.

Calculation formula for tumor volume:

Tumor volume=Major axis×Minor axis×Minor axis×0.5 [Expression 1]

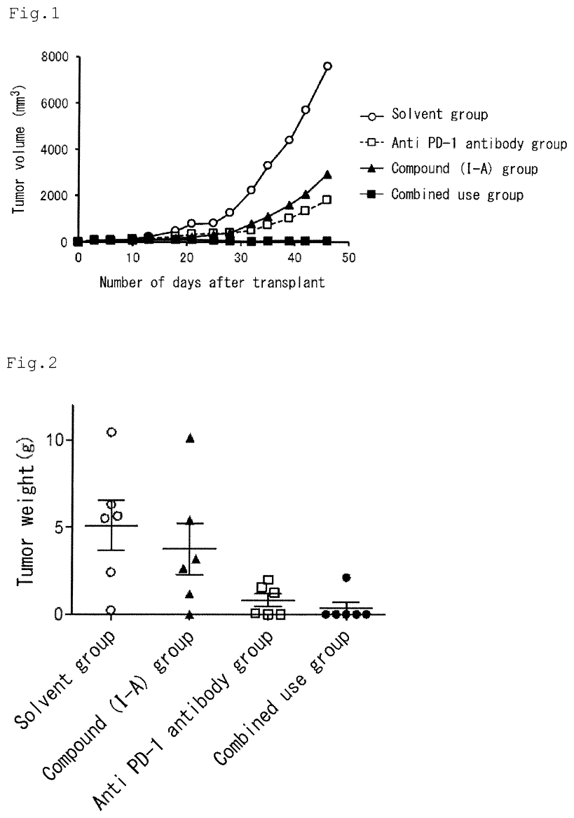

FIG. 1 shows the time courses of tumor volume in the different groups. As demonstrated in FIG. 1, the BTK inhibitor (I-A) alone suppressed tumor growth, and when being used further in combination with the anti-PD-1 antibody, exhibited significant tumor growth suppression/tumor regression effect. In the combinatorial use group, complete regression of tumor was found for five of the six mice with transplanted tumor. This result confirmed that the combination of a reversible BTK inhibitor and an immune checkpoint inhibitor according to the present invention provides superior antitumor effect, thus being useful in treatment of cancers.

### Example 2 Combined Effect of Reversible BTK Inhibitor and Anti-PD-1 Antibody in Allograft Mouse Model With Mouse B-Cell Lymphoma Cell Line A20

The antitumor effect of the compound according to the present invention was examined by using a syngeneic mouse tumor model with the mouse B-cell lymphoma cell line A20 (subcutaneous transplantation).

### (Culture of Cells for Use)

To RPMI-1640 medium (Life Technologies, No. A1049101), 10% FBS (HyClone), 0.05 mM 2-mercaptoethanol, and 1% penicillin streptomycin (NACALAI TESQUE, INC.) were added to prepare a cell culture medium (hereinafter, referred to as the medium 3). A20 cell (ATCC) were cultured with the medium 3 in a flask in a 5% CO₂ incubator.

### (Production of Cancer-Bearing Model)

The density of A20 cells was adjusted with the medium 2 to 5×10⁷ cells/mL; thus a cell preparation solution for transplantation was produced. Into the back of each BALB/cCrslc mouse (female, 8-week-old, Japan SLC, Inc.), 0.1 mL of the cell preparation solution for transplantation was subcutaneously transplanted. On day 3 after the transplantation of cancer cells, the cancer-bearing mice were grouped in such a manner that the means of tumor volume (see the calculation formula of Expression 1 in Example 1) approximated to each other.

### (Antitumor Effect Test for Test Substance)

Forced oral administration of 0.1 mL of the test substance or a solvent per 10 g body weight on that day (15 mg/kg) was performed to each mouse with transplanted cancer cells (six mice per group) twice per day (interval: 6 hours or longer) from day 3 to day 14 after the transplantation. No administration was performed on day 8 and 9. To each mouse, 0.1 mL of the antibody solution per 10 g body weight on that day (10 mg/kg) was intraperitoneally administered for an antibody administration group and a combined agents group, and the same amount of physiological saline was intraperitoneally administered for the other groups twice per week (four times in total). The mice were followed up until day 46 after the transplantation, and the tumor diameter of each mouse was measured to calculate the tumor volume by using the same formula as in Example 1 several times per week for evaluation of the antitumor effect.

In addition, tumor was excised from each mouse on day 46 after the transplantation, and the tumor weight was measured.

FIG. 2 shows the tumor weights in the different groups on day 46 after the transplantation.

As demonstrated in FIG. 2, the BTK inhibitor (I-A) when being used in combination with the anti-PD-1 antibody exhibited significant tumor growth suppression/tumor regression effect, and complete regression of tumor was found for five of the six mice with transplanted tumor. This result confirmed that the combination of a reversible BTK inhibitor and an immune checkpoint inhibitor according to the present invention provides superior antitumor effect, thus being useful in treatment of cancers.

### INDUSTRIAL APPLICABILITY

The combination of a reversible BTK inhibitor and an immune checkpoint inhibitor according to the present invention provides strong antitumor effect due to the combined effect, thus being useful for treating cancers.

1. A pharmaceutical composition for treating a cancer comprising a reversible BTK inhibitor and an immune checkpoint inhibitor in combination.

2. The pharmaceutical composition described in claim 1, wherein the reversible BTK inhibitor is an oxoisoquinoline derivative of the following formula (I) 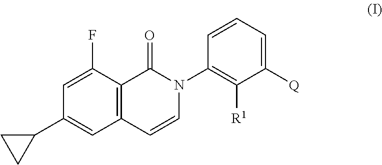
wherein R¹ is an optionally substituted lower alkyl group, Q is a structure selected from the following structures (a), (b) and (c); 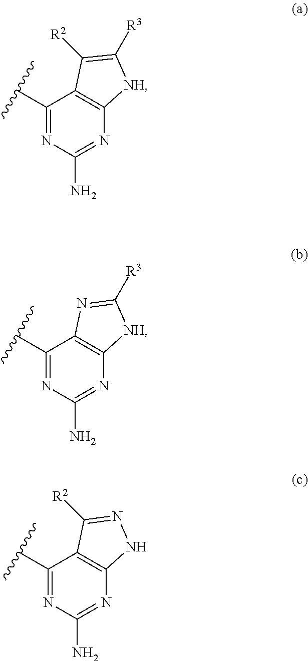
R² and R³ are independently a hydrogen atom, an optionally substituted lower alkyl group, an optionally substituted cycloalkyl group, an optionally substituted aryl group, an optionally substituted heteroaryl group or an optionally substituted heterocyclic group, or a pharmaceutically acceptable salt thereof.

3. The pharmaceutical composition described in claim 2, wherein Q is a structure (a), and R¹ is a hydroxymethyl group.

4. The pharmaceutical composition described in claim 1, wherein the reversible BTK inhibitor is an oxoisoquinoline derivative of the following formula (Ia) 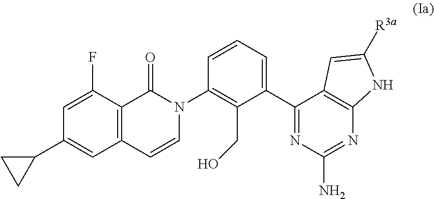
wherein R^{3a} is an optionally substituted tetrahydropyridyl group, or a pharmaceutically acceptable salt thereof.

5. The pharmaceutical composition described in claim 4, wherein the oxoisoquinoline derivative has a structure of Compound (I-A);
Compound (I-A): 2-(3-{2-amino-6-[1-(oxetan-3-yl)-1,2,3,6-tetrahydropyridin-4-yl]-7H -pyrrolo[2,3-d]pyrimidin-4-yl}-2-(hydroxymethyl)phenyl)-6-cyclopropyl-8-fluoroisoquinolin -1(2H)-one.

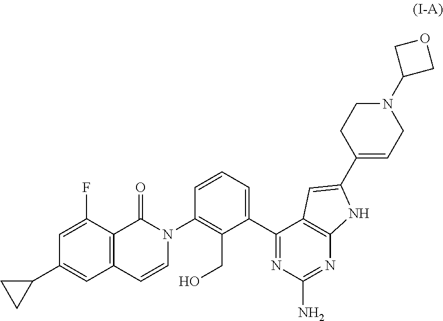 [SMILES: Nc1nc(-c2cccc(-n3ccc4cc(C5CC5)cc(F)c4c3=O)c2CO)c2cc(C3=CCN(C4COC4)CC3)[nH]c2n1]

6. The pharmaceutical composition described in claim 1, wherein the reversible BTK inhibitor is a triazine derivative of the following formula (II) 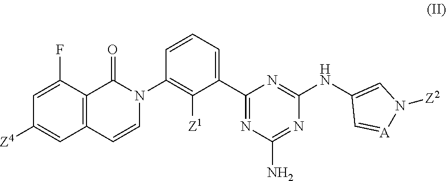
wherein Z¹ represents an optionally substituted lower alkyl group,
Z² represents a hydrogen atom or an optionally substituted lower alkyl group,
A represents a nitrogen atom or C-Z³,
Z³ represents a hydrogen atom, a cyano group, an optionally substituted acyl group, an optionally substituted sulfonyl group, or an optionally substituted carbamoyl group, Z⁴ represents an optionally substituted lower alkyl group, an optionally substituted cycloalkyl group, or a pharmaceutically acceptable salt thereof.

7. The pharmaceutical composition described in claim 6, wherein Z¹ is a hydroxymethyl group.

8. The pharmaceutical composition described in claim 6, wherein the triazine derivative has a structure of Compound (II-A);
Compound (II-A): 2-(3-{4-Amino-6-[(1-methyl-1H-pyrazol-4-yl)amino]-1,3,5-triazin-2-yl}-2-(hydroxymethyl)phenyl)-6-cyclopropyl-8-fluoroisoquinolin-1(2H)-one.

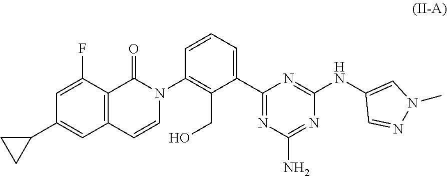 [SMILES: Cn1cc(Nc2nc(N)nc(-c3cccc(-n4ccc5cc(C6CC6)cc(F)c5c4=O)c3CO)n2)cn1]

9. The pharmaceutical composition described in claim 1, wherein the immune checkpoint inhibitor is an inhibitor to immune checkpoint molecules selected from the group consisting of PD-1, PD-L1, PD-L2, CTLA-4, LAG-3, TIM3, BTLA, B7H3, B7H4, CD160, CD39, CD73, A2aR, KIR, VISTA, IDO1, ArginaseI, TIGIT and CD115.

10. The pharmaceutical composition described in claim 9 wherein the immune checkpoint inhibitor is an anti-PD-1 antibody.

11. The pharmaceutical composition described in claim 9, wherein the immune checkpoint inhibitor is an anti-PD-L1 antibody.

12. The pharmaceutical composition described in claim 9, wherein the immune checkpoint inhibitor is an anti-PD-L2 antibody.

13. The pharmaceutical composition described in claim 9, wherein the immune checkpoint inhibitor is an anti-CTLA-4 antibody.

14. Use of a compound (I) and an immune checkpoint inhibitor in manufacturing the pharmaceutical composition described in claim 1.

15. Method for treating a cancer characterized in using the pharmaceutical composition described in claim 1.
